(19) United States
(12) Patent Application Publication
(10) Pub. No.: US 2008/0033264 A1
(43) Pub. Date: Feb. 7, 2008

(54) Pulsoximetry Measuring Device

(76) Inventors: Torsten Lonneker-Lammers, Gross Sarau (DE); Torsten Hertz, Lubeck (DE)

Correspondence Address: ALIX YALE & RISTAS LLP, 750 MAIN STREET, SUITE 1400, HARTFORD, CT, 06103, US

(21) Appl. No.: 11/791,433

(22) PCT Filed: Nov. 23, 2005
(86) PCT No.: PCT/EP05/12550

§ 371 Date: May 23, 2007

(30) Foreign Application Priority Data
Nov. 23, 2004 (DE) 10 2004 056 587.2

(51) Int. Cl. A61B 5/1455

(52) U.S. Cl. 600/323

(57) ABSTRACT

Disclosed is a pulsoximetry measuring device comprising a pulsoximetry sensor and a pulsoximetry module for evaluating and displaying the sensor signals. The device is characterized in that the pulsoximetry module is provided with a shield which is grounded only at one point while each signal path is equipped with a rejection filter having a narrow passage area.

### BACKGROUND

The invention relates to a pulsoximetry measuring device having a pulsoximeter sensor and a pulsoximeter module for evaluating and displaying the signals of the sensor.

The detection and monitoring of vital parameters in the case of new born and prematurely born patients both at the intensive station and during transportation constitutes a basic requirement in everyday hospital practice. Consequently, there are on the market a large number both of portable and of fixed patient monitors, in the specific, so-called pulsoximeters, with the aid of which the oxygen saturation and heart rate of the patient can be determined non-invasively.

The selection of available pulsoximeters is restricted in the field of diagnostics using magnetic resonance (MR resonance). One reason for this is that the interference-free operation of electronic equipment in the direct environment of nuclear magnetic resonance tomographs is impossible without particular measures, because of the strong electromagnetic fields. Equipment therefore frequently exhibits awkward handling, since an attempt is predominantly made, through the introduction of long connecting lines (electrical or optical), on the one hand, to position the sensor near the patient and, on the other hand, to position the electronic evaluation and display unit as far as possible from the tomograph.

The measurement principle of pulsoximetry is based on the wavelength dependent optical perfusion of the blood vessels located under the skin. The differences in power and features to be found in the case of the pulsoximeters offered on the market are to be ascribed to different algorithms for signal processing, and are based on wide experience and a knowledge base in the field of pulsoximetry. Consequently, in addition to stand alone equipment, some manufacturers also offer so-called OEM modules that to some extent constitute the core of the acquisition and processing of measured values, and are therefore eminently suitable for installation in other medical equipment. However, such equipment cannot be used in the vicinity of nuclear magnetic resonance tomographs without the use of the abovementioned long connecting lines so that the sensitive pulsoximetry module is sufficiently far away from the static magnetic fields and electromagnetic high frequency measuring fields of the nuclear magnetic resonance tomograph. Because of the strong fields, it has therefore not so far been possible to arrange the pulsoximetry module near the patient and the nuclear magnetic resonance tomograph, and this palpably signifies disadvantages for the examination and treatment of the patient.

### SUMMARY

A pulsoximetry measuring device can be integrated in an existing, MR capable medical unit, for example in a patient monitor or an incubator.

The pulsoximeter module is provided with a shield, in that the shield is grounded only at one point, and in that each signal path is provided with a rejection filter having a narrowband passband.

A combination of three measures is aimed at integrating in a medical unit an OEM module offered on the market. An important role is played here from the point of view of metrology by the fact that no significant interference with regard to imaging or measurement accuracy occurs between MRT and pulsoximetry. Even more important, however, is the exclusion of any sort of endangerment of patient and user with regard to heating of sensor or cable because of the coupling, unavoidable in MRT, of high frequency energy and the production of eddy currents caused by magnetic fields that vary in time and space.

Consequently, it is a fundamental measure to shield all the participating components and their connections from the very first.

Each enclosing shield ends at a grounding point; the presence of grounding loops impairs imaging and measurement accuracy and is therefore avoided.

Filtering the signals between the sensor and OEM module is the third, and most important measure.

In one advantageous embodiment, the filter has an LC element (passive filter of 2nd order).

The pass frequency of the narrowband filter advantageously lies in the range from 0.1 to 15 MHz. The pass frequency and the signal frequencies of the pulsoximeter do not overlap then, since the magnetic field strength of 1.5 T is the Larmor frequency of the protons 63.9 MHz.

It is yet more advantageous when the pass frequency of the narrowband filter lies in the range from 0.1 to 8 MHz.

In particular, the pass frequency of the narrowband filter can be substantially less than 10 MHz.

In a particularly advantageous embodiment, its evaluation unit can be integrated in the control electronics of an incubator, and is to be supplied by the latter with power.

### BRIEF DESCRIPTION OF THE DRAWINGS

FIG. 1 shows a schematic of a signal path from source to sink;

FIG. 2 shows the signal path of FIG. 1, with a filter;

FIG. 3 shows the frequency response of the signals of a nuclear magnetic resonance tomograph with a magnetic field strength of 1.5 T; and

FIG. 4 shows a schematic of the design of a pulsoximetry measuring device.

### DETAILED DESCRIPTION

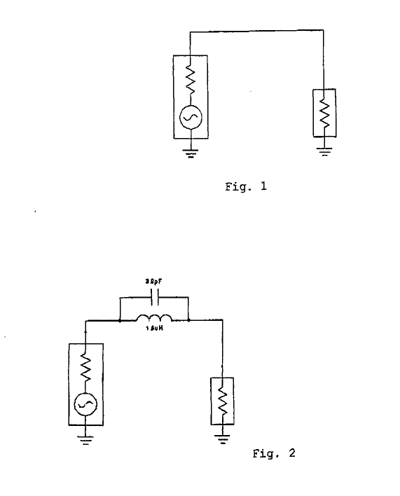

As FIG. 1 shows, each signal is guided between source (Q) and sink (S) along a path (as a rule, an electric cable). The source is represented on the left, and the sink on the right. A minimum of four signal paths are required between sensor and OEM module in the exemplary pulsoximetry module:

| | OEM | |
|---|---|---|
| Sensor | module | Description |
| S | Q | Transmit light emitting diodes (+) pole |
| S | Q | Transmit light emitting diodes (−) pole |
| Q | S | Receive photodiode (+) pole |
| Q | S | Receive photodiode (−) pole |

The frequency spectra applied by the MRT are very narrowband in the respective equipment class, and so looping in a selective higher order rejection filter along each signal path between sensor and evaluation unit not only minimizes the abovementioned interference, but greatly reduces both HF coupling and eddy currents.

Such a rejection filter can be implemented in a simple and yet effective way as an LC element (passive filter of 2nd order), as is shown in FIG. 2. In the case of pulsoximetry, the useful frequency range (<<10 MHz) is far enough from that of MRT (42 . . . 130 MHz) for filtering not to cause any negative side effects.

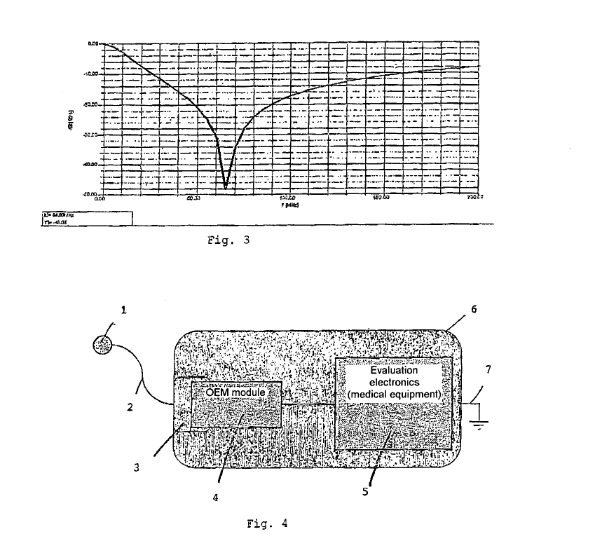

FIG. 3 shows the frequency response in the case of the use of the rejection filter according to FIG. 2. The resonant frequency was tuned for an MRT system with a 1.5 T magnetic field strength which corresponds to a Lamor frequency of 63.9 MHz. In this range, the insertion loss is better than 40 dB.

This filtering is present on each of the four above-named signal paths between sensor and OEM module. The principle design of the pulsoximetry measuring device is shown in FIG. 4.

A sensor 1 is connected via a shielded cable 2 and filter 3 to the OEM module 4, which is connected, in turn, to an evaluation electronics 5. The filter 3, OEM module 4 and evaluation electronics 5 are arranged inside a shield housing 6 that is grounded at one point at 7.

1. A pulsoximetry measuring device having a pulsoximeter sensor and a pulsoximeter module for evaluating and displaying the signals of the sensor, characterized in that the pulsoximeter module is provided with a shield, in that the shield is grounded only at one point, and in that each signal path is provided with a rejection filter having a narrowband passband.

2. The measuring device as claimed in claim 1, characterized in that the rejection filter has an LC element.

3. The measuring device as claimed in claim 1, characterized in that the pass frequency of the narrowband filter lies in the range from 0.1 to 15 MHz.

4. The measuring device as claimed in claim 3, characterized in that the pass frequency of the narrowband filter lies in the range from 0.1 to 8 MHz.

5. The measuring device as claimed in claim 3, characterized in that the pass frequency of the narrowband filter is substantially lower than 10 MHz.

6. The measuring device as claimed in claim 1, characterized in that its evaluation unit is integrated in control electronics of an incubator, and is supplied by the latter with power.

7. The measuring device as claimed in claim 1, characterized in that the rejection filters are arranged in the vicinity of plug-in connectors.

8. The measuring device as claimed in claim 1, characterized in that the rejection filters are arranged in the shield.

9. The measuring device as claimed in claim 2, characterized in that the pass frequency of the narrowband filter lies in the range from 0.1 to 15 MHz.

10. The measuring device as claimed in claim 9, characterized in that the pass frequency of the narrowband filter lies in the range from 0.1 to 8 MHz.

11. The measuring device as claimed in claim 9, characterized in that the pass frequency of the narrowband filter is substantially lower than 10 MHz.

12. The measuring device as claimed in claim 2, characterized in that its evaluation unit is integrated in control electronics of an incubator, and is supplied by the latter with power.

13. The measuring device as claimed in claim 3, characterized in that its evaluation unit is integrated in control electronics of an incubator, and is supplied by the latter with power.

14. The measuring device as claimed in claim 4, characterized in that its evaluation unit is integrated in control electronics of an incubator, and is supplied by the latter with power.

15. The measuring device as claimed in claim 5, characterized in that its evaluation unit is integrated in control electronics of an incubator, and is supplied by the latter with power.

16. The measuring device as claimed in claim 2, characterized in that the rejection filters are arranged in the vicinity of plug-in connectors.

17. The measuring device as claimed in claim 3, characterized in that the rejection filters are arranged in the vicinity of plug-in connectors.

18. The measuring device as claimed in claim 4, characterized in that the rejection filters are arranged in the vicinity of plug-in connectors.

19. The measuring device as claimed in claim 5, characterized in that the rejection filters are arranged in the vicinity of plug-in connectors.

20. The measuring device as claimed in claim 6, characterized in that the rejection filters are arranged in the vicinity of plug-in connectors.
